(19) United States
(12) Patent Application Publication
(10) Pub. No.: US 2004/0115220 A1
(43) Pub. Date: Jun. 17, 2004

(54) Vaccine

(76) Inventor: Carlota Vinals Y De Bassols, Rixensart (BE)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/415,389

(22) PCT Filed: Oct. 24, 2001
(86) PCT No.: PCT/EP01/12392

(30) Foreign Application Priority Data
Oct. 27, 2000 (GB) 0026334.3

(51) Int. Cl. C07K 14/02; A61K 39/29

(52) U.S. Cl. 424/189.1; 530/350

(57) ABSTRACT

The present invention relates to the provision of novel medicaments for the treatment, prevention or amelioration of allergic disease. In particular, the novel medicaments are epitopes or mimotopes derived from IgE. The novel regions presented may be the target for both passive and active immunoprophylaxis or immunotherapy. The invention further relates to methods for production of the medicaments, pharmaceutical compositions containing them and their use in medicine. Also forming an aspect of the present invention are ligands, especially monoclonal antibodies, which are capable of binding the IgE regions of the present invention, and their use in medicine as passive immunotherapy or immunoprophylaxis.

The present invention relates to the provision of novel medicaments for the treatment, prevention or amelioration of allergic disease. In particular, the novel medicaments are epitopes or mimotopes derived from IgE. These novel regions may be the target for both passive and active immunoprophylaxis or immunotherapy. The invention further relates to methods for production of the medicaments, pharmaceutical compositions containing them and their use in medicine. Also forming an aspect of the present invention are ligands, especially monoclonal antibodies, which are capable of binding the IgE regions of the present invention, and their use in medicine as passive immunotherapy or immunoprophylaxis.

In an allergic response, the symptoms commonly associated with allergy are brought about by the release of allergic mediators, such as histamine, from immune cells into the surrounding tissues and vascular structures. Histamine is normally stored in mast cells and basophils, until such time as the release is triggered by interaction with allergen specific IgE The role of IgE in the mediation of allergic responses, such as asthma, food allergies, atopic dermatitis, type-I hypersensitivity and allergic rhinitis, is well known. On encountering an antigen, such as pollen or dust mite allergens, B-cells commence the synthesis of allergen specific IgE. The allergen specific IgE then binds to the FcεRI receptor (the high affinity IgE receptor) on basophils and mast cells. Any subsequent encounter with allergen leads to the triggering of histamine release from the mast cells or basophils, by cross-linking of neighbouring IgE/FcεRI complexes (Sutton and Gould, Nature, 1993, 366: 421-428; EP0 477 231 B1).

IgE, like all immunoglobulins, comprises two heavy and two light chains. The ε heavy chain consists of five domains: one variable domain (VH) and four constant domains (Cε1 to Cε4). The molecular weight of IgE is about 190,000 Da, the heavy chain being approximately 550 amino acids in length. The structure of IgE is discussed in Padlan and Davis (Mol. Immunol., 23, 1063-75, 1986) and Helm et al., (2IgE model structure deposited Feb. 10, 1990 with PDB (Protein Data Bank, Research Collabarotory for Structural Bioinformatics; http:\pdb-browsers.ebi.ac.uk)). Each of the IgE domains consists of a squashed barrel of seven anti-parallel strands of extended (β-) polypeptide segments, labelled a to f, grouped into two β-sheets. Four β-strands (a, b, d & e) form one sheet that is stacked against the second sheet of three strands (c, f & g). The shape of each β-sheet is maintained by lateral packing of amino acid residue side-chains from neighbouring anti-parallel strands within each sheet (and is further stabilised by main-chain hydrogen-bonding between these strands). Loops of residues, forming non-extended (non-β-) conformations, connect the anti-parallel β-strands, either within a sheet or between the opposing sheets. The connection from strand a to strand b is labelled as the A-B loop, and so on. The A-B and d-e loops belong topologically to the four-stranded sheet, and loop f-g to the three-stranded sheet. The interface between the pair of opposing sheets provides the hydrophobic interior of the globular domain. This water-inaccessible, mainly hydrophobic core results from the close packing of residue side-chains that face each other from opposing β-sheets.

In the past, a number of passive or active immunotherapeutic approaches designed to interfere with IgE-mediated histamine release mechanism have been investigated. These approaches include interfering with IgE or allergen/IgE complexes binding to the FcεRI or FcεRII (the low affinity IgE receptor) receptors, with either passively administered antibodies, or with passive administration of IgE derived peptides to competitively bind to the receptors. In addition, some authors have described the use of specific peptides derived from IgE in active immunisation to stimulate histamine release inhibiting immune responses.

In the course of their investigations, previous workers in this field have encountered a number of considerations, and problems which have to be taken into account when designing new anti-allergy therapies. One of the most dangerous problems revolves around the involvement of IgE cross-linking in the histamine release signal. It is most often the case that the generation of anti-IgE antibodies during active vaccination, are capable of triggering histamine release per se, by the cross-linking of neighbouring IgE-receptor complexes in the absence of alergen. This phenomenon is termed anaphylactogenmcity. Indeed many commercially available anti-IgE monoclonal antibodies which are normally used for IgE detection assays, are anaphylactogenic, and consequently useless and potentially dangerous if administered to a patient.

Whether or not an antibody is anaphylactogenic, depends on the location of the target epitope on the IgE molecule. However, based on the present state of knowledge in this area, and despite enormous scientific interest and endeavour, there is little or no predictability of what characteristics any antibody or epitope may have and whether or not it might have a positive or negative clinical effect on a patient.

Therefore, in order to be safe and effective, the passively administered, or vaccine induced, antibodies must bind in a region of IgE which is capable of interfering with the histamine triggering pathway, without being anaphylactic per se. The present invention achieves all of these aims and provides medicaments which are capable of raising non-anaphylactic antibodies which inhibit histamine release. These medicaments may form the basis of an active vaccine or be used to raise appropriate antibodies for passive immunotherapy) or may be passively administered themselves for a therapeutic effect.

Much work has been carried out by those skilled in the art to identify specific anti-IgE antibodies which do have some beneficial effects against IgE-mediated allergic reaction (WO 90/15878, WO 89/04834, WO 93/05810). Attempts have also been made to identify epitopes recognised by these useful antibodies, to create peptide mimotopes of such epitopes and to use those as immunogens to produce anti-IgE antibodies.

WO 97/31948 describes an example of this type of work, and further describes IgE peptides from the Cε3 and Cε4 domains conjugated to carrier molecules for active vaccination purposes. These immunogens-may be used in vaccination studies and are said to be capable of generating antibodies which, subsequently inhibit histamine release in vivo. In this work, a monoclonal antibody (BSW17) was described which was said to be capable of binding to IgE peptides contained within the Cε3 domain which are useful for active vaccination purposes.

EP 0 477 231 B1 describes immunogens derived from the Cε4 domain of IgE (residues 497-506, also known as the Stanworth decapeptide), conjugated to Keyhole Limpet Haemocyanin (KLH) used in active vaccination immunoprophylaxis. WO 96/14333 is a continuation of the work described in EP 0 477 231 B1.

WO 99/67293; WO 00/50460 and WO 00/50461 all describe IgE peptide immunogens for active immunotherapy of allergy by vaccination.

Other approaches are based on the identification of peptides derived from Cε3 or Cε4, which themselves compete for IgE binding to the high or low affinity receptors on basophils or mast cells (WO 93/04173, WO 98/24808, EP 0 303 625 B1, EP 0 341 290).

The present invention is the identification of novel sequences of IgE which are used in active or passive immunoprophylaxis or therapy. These sequences have not previously been associated with anti-allergy treatments. The present invention provides peptides, per se, that incorporate specific isolated epitopes from continuous portions of IgE which have been identified as being surface exposed, and further provides mimotopes of these newly identified epitopes. These peptides or mimotopes may be used alone in the treatment of allergy, or may be used vaccines to induce auto anti-IgE antibodies during active immunoprophylaxis or immunotherapy of allergy to limit, reduce, or eliminate allergic symptoms in vaccinated subjects.

Surprisingly, the anti-IgE antibodies induced by the peptides of the present invention are non-anaphylactogenic and are capable of blocking IgE-mediated histamine release from mast cells and basophils.

The regions of human IgE which are peptides of the present invention, and which may serve to provide the basis for peptide modification are:

TABLE 1

| | | Location sequence | SEQ |
|---|---|---|---|
| | | and IgE | ID |
| Peptide | Sequence | Domain | NO. |
| Helix3 | DSNPRGVSA | Cε2-3 linker | 1 |
| Carl4 | LVVDLAPSKGTVN | 2-90N mimotope | 2 |
| Carl5 | KQRNGTL | P5 mimotope | 3 |
| Carl6 | EEKQRNGTLTV | P5 mimotope | 4 |
| Carl7 | HPHLPR | P7 mimotope | 5 |
| Carl8 | THPHLPRA | P7 mimotope | 6 |
| Carl9 | VTHPHLPRAL | P7 mimotope | 7 |
| Carl10 | RVTHPHLPRALM | P7 mimotope | 8 |
| Carl11 | XRVTHPHLPRALMR | P7 mimotope | 9 |
| Carl12 | QXRVTHPHLPRALMRS | P7 mimotope | 10 |
| Carl13 | YQXRVTHPHLPRALMRST | P7 mimotope | 11 |
| Carl14 | PEWPGSRDKR | P8 mimotope | 12 |
| BOA | CDSNPRGVSAADSNPRGVSC | cyclised Helix 3 multi peptide | 13 |
| Carl15 | CLVVDLAPSKGTVNC | 2-90N mimotope | 14 |
| Carl16 | CKQRNGTLC | P5 mimotope | 15 |
| Carl17 | CEEKQRNGTLTVC | P5 mimotope | 16 |
| Carl18 | CHPHLPRC | P7 mimotope | 17 |
| Carl19 | CTHPHLPRAC | P7 mimotope | 18 |
| Carl20 | CVTHPHLPRALC | P7 mimotope | 19 |
| Carl21 | CRVTHPHLPRALMC | P7 mimotope | 20 |
| Carl22 | CXRVTHPHLPRALMRC | P7 mimotope | 21 |
| Carl23 | CQXRVTHPHLPRALMRSC | P7 mimotope | 22 |
| Carl24 | CYQXRVTHPHLPRALMRSTC | P7 mimotope | 23 |
| Carl25 | CPEWPGSRDKRC | P8 mimotope | 24 |
| Carl26 | CRQRNGTLC | P5 mimotope | 25 |
| Carl27 | CEERQRNGTLTVC | P5 mimotope | 26 |
| Carl28 | CMRVTHPHLPRALMRC | P7 mimotope | 27 |
| Carl29 | CQMRVTHPHLPRALMRSC | P7 mimotope | 28 |
| Carl30 | CYQMRVTHPHLPRALMRSTC | P7 mimotope | 29 |
| Carl31 | RQRNGTL | P5 mimotope | 30 |
| Carl32 | EERQRNGTLTV | P5 mimotope | 31 |
| Carl33 | MRVTHPHLPRALMR | P7 mimotope | 32 |
| Carl34 | QMRVTHPHLPRALMRS | P7 mimotope | 33 |
| Carl35 | YQMRVTHPHLPRALMRST | P7 mimotope | 34 |

Of these, particularly preferred peptides are selected from the following list:

Cys (359)-LVVDLAPSKGTVN-(371)Cys

Cys-(391)-KQRNGTL-(397)-Cys

Cys-(389)-EEKQRNGTLTV-(398)-Cys

Cys-(422)-HPHLPR-(427)-Cys

Cys-(421)-THPHLPRA-(428)-Cys

Cys-(420)-VTHPHLPRAL-(429)Cys

Cys-(419)-RVTHPHLPRALM-(430)-Cys

Cys-(418)-cRVTHPHLPRALMR-(431)-Cys

Cys-(417)-QcRVTHPHLPRALMRS-(430)-Cys

Cys-(416)-YQcRVTHPHLPRALMRST-(431)-Cys [particularly preferred]

Cys-(451)-PEWPGSRDKR-(460)-Cys

wherein the small letter c in the above peptide sequences (or X in Table 1) denotes a natural cysteine, which may optionally be substituted with any other amino acid residue, but in this respect a substitution with Methionine is preferred. Preferably the substituted residue is not serine. The numbers in brackets denote the amino acid position within the IgE molecule. Immunogens comprising these peptides conjugated to Protein D or BSA, or expressed within HepB core protein form preferred aspects of the present invention.

Peptides that incorporate these epitopes form a preferred aspect of the present invention. Accordingly, peptides of the present invention may be longer than any peptides listed herein, as such peptides of the present invention may comprise the listed peptides, which may result from the addition of amino acids onto either or both ends of the listed peptide. In this regard, the additional residues may be derived from the natural sequence of IgE or not. The peptides may also be shorter than the listed peptides, by the removal of amino acids from either end. In both of these aspects of the invention the addition or removal of residues concerns preferably less than 10 amino acids, more preferably less than 5 amino acids, more preferably less than 3 amino acids, and most preferably concerns 2 amino acids or less, which may be added to or removed from either end of the listed peptides.

Mimotopes which have the same characteristics as these epitopes, and immunogens comprising such mimotopes which generate an immune response which cross-react with the IgE epitope in the context of the IgE molecule, also form part of the present invention.

The present invention, therefore, includes isolated peptides encompassing these IgE epitopes themselves, and any mimotope thereof. The meaning of mimotope is defined as an entity which is sufficiently similar to the native IgE epitope so as to be capable of being recognised by antibodies which recognise the native IgE epitope; (Gheysen, H. M., et al., 1986, Synthetic peptides as antigens. Wiley, Chichester, Ciba foundation symposium 119, p130-149; Gheysen, H. M.; 1986, Molecular Immunology, 23,7, 709-715); or are capable of raising antibodies, when coupled to a suitable carrier, which antibodies cross-react with the native IgE epitope.

The mimotopes of the present invention mimic the surface exposed regions of the IgE structure, however, within those regions the dominant aspect is thought by the present inventors to be those regions within the surface exposed area which correlate to a loop structure. The structure of the domains of IgE are described in “Introduction to protein Structure” (page 304, 2^{nd} Edition, Branden and Tooze, Garland Publishing, New York,. ISBN 0 8153 2305-0) and take the form a β-barrel made up of two opposing anti-parallel β-sheets (see FIG. 8). The mimotopes may comprise, therefore, a loop with N or C terminal extensions which may be the natural amino acid residues from neighbouring sheets, and they may also comprise Helix 3 the Cε2-3 linker. As examples of this, P100 contains the A-B loop of Cε3; Carl4 contains the B-C loop of Cε3; Carl5 contains the D-E loop of Cε3; Carl7 contains the F-G loop of Cε3; P8 contains the A-B loop of Cε4; P5 contains the C-D loop of Cε3 and P110 contains the C-D loop of Cε4. Accordingly, mimotopes of these loops form an aspect of the present invention.

The most preferred loops for formulation into vaccines of the present invention are the B-C loop of Cε3, the D-E loop of Cε3 and the F-G loop of Cε3. Also forming a particularly preferred peptide of the present invention is Cε2-3 linker. As such, the peptides; and immunogens comprising them, may be used alone. Additionally, combination vaccines comprising these most preferred immunogens are especially useful in the treatment of allergy.

Peptide mimotopes of the above-identified IgE epitopes may be designed for a particular purpose by addition, deletion or substitution of elected amino acids. As such the peptide immunogens of the present invention may be altered as a result from the addition, deletion, or substitution of any residue of the peptide sequences listed herein. When the alteration is an addition or a substitution, it may involve a natural or non-natural amino acid, and may involve the addition of amino acid residues derived from the corresponding region of IgE. Alterations of the peptide sequences preferably involve less than 10 amino acid residues, more preferably less than 5 residues, more preferable less than 3 residues, and most preferably involves 2 amino acid residues or less. Thus, the peptides of the present invention may be modified for the purposes of ease of conjugation to a protein carrier, for example by the addition of a terminal cysteine or add a linker sequence, such as a double Glycine head or tail, or a linker terminating with a lysine residue. Alternatively, the addition or substitution of a D-stereoisomer form of one or more of the amino acids may be performed to create a beneficial derivative, for example to enhance stability of the peptide. Those skilled in the art will realise that such modified peptides, or mimotopes, could be a wholly or partly non-peptide mimotope wherein the constituent residues are not necessarily confined to the 20 naturally occurring amino acids.

Preferably the peptides are cyclised by techniques known in the art to constrain the peptide into a conformation that closely resembles its shape when the peptide sequence is in the context of the whole IgE molecule. A preferred method of cyclising a peptide comprises the addition of a pair of cysteine residues to allow the formation of a disulphide bridge.

The peptide mimotopes may also be retro sequences of the natural IgE sequences, in that the sequence orientation is reversed; or alternatively the sequences may be entirely or at least in part comprised of D-stereoisomer amino acids (inverso sequences). Also, the peptide sequences may be retro-inverso in character, in that the sequence orientation is reversed and the amino acids are of the D-stereoisomer form. Such retro or retro-inverso peptides have the advantage of being non-self, and as such may overcome problems of self-tolerance in the immune system.

Multi-peptide immunogens (preferably cyclised/constrained as described above) may be formed from the listed peptides sequences or mimotopes thereof, which may be advantageous in the induction of an immune response. For example Helix 3 is an example of peptide which can be constructed into a dimeric peptide repeat, such that there is a repeating peptide epitope contained within the sequence. For example, BOA comprises a Helix 3 tandem repeat with the addition of two cysteines at each end to constrain the dimer at each end, thereby limiting the structural freedom of the peptide. In this way each monomeric unit may be constrained in structure, yet still have the possibility to fold into native-like extended structures (for instance helix-turns). Such multi-peptide immunogens are preferred immunogens of the present invention.

Alternatively, peptide mimotopes may be identified using antibodies which are capable themselves of binding to the IgE epitopes of the present invention using techniques such as phage display technology (EP 0 552 267 B1). This technique, generates a large number of peptide sequences which mimic the structure of the native peptides and are, therefore, capable of binding to anti-native peptide antibodies, but may not necessarily themselves share significant sequence homology to the native IgE peptide. This approach may have significant advantages by allowing the possibility of identifying a peptide with enhanced immunogenic properties (such as higher affinity binding characteristics to the IgE receptors or anti-IgE antibodies, or being capable of inducing polyclonal immune response which binds to IgE with higher affinity), or may overcome any potential self-antigen tolerance problems which may be associated with the use of the native peptide sequence. Additionally this technique allows the identification of a recognition pattern for each native-peptide in terms of its shared chemical properties amongst recognised mimotope sequences.

Alternatively, peptide mimotopes may be generated with the objective of increasing the immunogenicity of the peptide by increasing its affinity to the anti-IgE peptide polyclonal antibody, the effect of which may be measured by techniques known in the art such as (Biocore experiments). In order to achieve this the peptide sequence may be electively changed following the general rules:

To maintain the structural constraints, prolines and glycines should not, be replaced

Other positions can be substituted by an amino acid that has similar physicochemical properties.

As such, each amino acid residue can be replaced by the amino acid that most closely resembles that amino acid.

The present invention, therefore, provides novel epitopes, and mimotopes thereof, and their use in the manufacture of pharmaceutical compositions for the prophylaxis or therapy of allergies. Immunogens comprising at least one of the epitopes or mimotopes of the present invention and carrier molecules are also provided for use in vaccines for the immunoprophylaxis or therapy of allergies. Accordingly, the epitopes, mimotopes, or immunogens of the present invention are provided for use in medicine, and in the medical treatment or prophylaxis of allergic disease.

It is envisaged that the mimotopes of the present invention will be of a small size, such that they mimic a region selected from the whole IgE domain in which the native epitope is found. Peptidic mimotopes, therefore, should be less than 100 amino acids in length, preferably shorter than 75 amino acids, more preferably less than 50 amino acids, and most preferable within the range of 4 to 25 amino acids long. Specific examples of preferred peptide mimotopes are P14and P11, which are respectively 13 and 23 amino acids long. Non-peptidic mimotopes are envisaged to be of a similar size, in terms of molecular volume, to their peptidic counterparts.

It will be apparent to the man skilled in the art which techniques may be used to confirm the status of a specific construct as a mimotope which falls within the scope of the present invention. Such techniques include, but are not restricted to, the following. The putative mimotope can be assayed to ascertain the immunogenicity of the construct, in that antisera raised by the putative mimotope cross-react with the native IgE molecule and are also functional in blocking allergic mediator release from allergic effector cells. The specificity of these responses can be confirmed by competition experiments by blocking the activity of the antiserum with the mimotope itself or the native IgE, and/or specific monoclonal antibodies that are known to bind the epitope within IgE.

In one embodiment of the present invention at least one IgE epitope or mimotope are linked to carrier molecules to form immunogens for vaccination protocols, preferably wherein the carrier molecules are not related to tentative IgE molecule. The mimotopes may be linked via chemical covalent conjugation or by expression of genetically engineered fusion partners, optionally via a linker sequence. As one embodiment, the peptides of the present invention are expressed in a fusion molecule with the fusion partner, wherein the peptide sequence is found within the primary sequence of the fusion partner.

The covalent coupling of the peptide to the immunogenic carrier can be carried out in a manner well known in the art. Thus, for example, for direct covalent coupling it is possible to utilise a carbodiimide, glutaraldehyde or (N-[γ-maleimidobutyryloxy] succinimide ester, utilising common commercially available heterobifunctional linkers such as CDAP and SPDP (using manufacturers instructions). After the coupling reaction, the immunogen can easily be isolated and purified by means of a dialysis method, a gel filtration method, a fractionation method etc.

In a preferred embodiment the present inventors have found that peptides, particularly cyclised peptides may be conjugated to the carrier by preparing Acylhydrazine peptide derivatives.

The peptides/protein carrier constructs can be produced as follows. Acylhydrazine peptide derivatives can be prepared on the solid phase as shown in the following scheme 1 Solid Phase Peptide Synthesis:

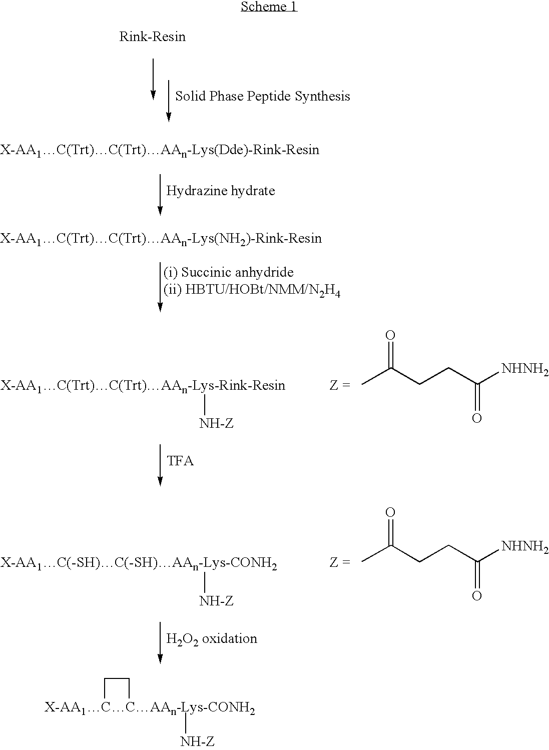

These peptide derivatives can be readily prepared using the well-known ‘Fmoc’ procedure, utilising either polyamide or polyethyleneglycol-polystyrene (PEG-PS) supports in a fully automated apparatus, through techniques well known in the art [techniques and procedures for solid phase synthesis are described in ‘Solid Phase Peptide Synthesis: A Practical Approach’ by E. Atherton and R. C. Sheppard, published by IRL at Oxford University Press (1989)]. Acid mediated cleavage afforded the linear, deprotected, modified peptide. This could be readily oxidised and purified to yield the disulphide-bridged modified epitope using methodology outlined in ‘Methods in Molecular Biology, Vol. 35: Peptide Synthesis Protocols (ed. M. W. Pennington and B. M. Dunn), chapter 7, pp91-171 by D. Andreau et al.

The peptides thus synthesised can then be conjugated to protein carriers using the following technique:

Introduction of the aryl aldehyde functionality utilised the succinimido active ester (BAL—OSu) prepared as shown in scheme 2 (see WO 98/17628 for further details). Substitution of the amino functions of a carrier eg BSA (bovine serum albumin) to ˜50% routinely give soluble modified protein. Greater substitution of the BSA leads to insoluble constructs. BSA and BAL—OSu were mixed in equimolar concentration in DMSO/buffer (see scheme) for 2 hrs. This experimentally derived protocol gives ˜50% substitution of BSA as judged by the Fluorescamine test for free amino groups in the following Scheme 2/3—Modified Carrier Preparation:

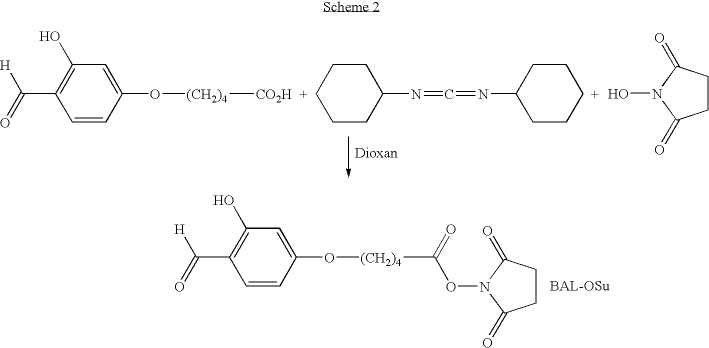

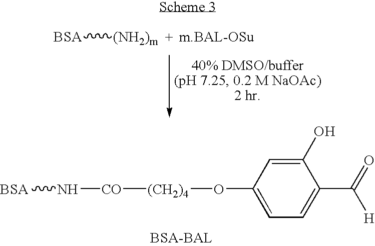

Simple combination of modified peptide and derivatised carrier affords peptide carrier constructs readily isolated by dialysis—Scheme 4—Peptide/carrier conjugate:

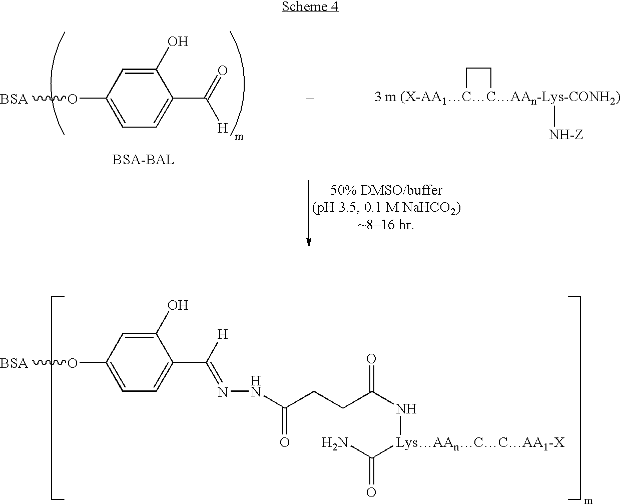

The types of carriers used in the immunogens of the present invention will be readily known to the man skilled in the art. The function of the carrier is to provide cytokine help in order to help induce an immune response against the IgE peptide. A non-exhaustive list of carriers which may be used in the present invention include: Keyhole limpet Haemocyanin (KLH), serum albumins such as bovine serum albumin (BSA), inactivated bacterial toxins such as tetanus or diptheria toxins (TT and DT) or CRM197, or recombinant fragments thereof (for example, Domain 1 of Fragment C of TT, or the translocation domain of DT), or the purified protein derivative of tuberculin (PPD). Alternatively the mimotopes or epitopes may be directly conjugated to liposome carriers, which may additionally comprise immunogens capable of providing T-cell help. Preferably the ratio of mimotopes to carrier is in the order of 1:1 to 20:1, and preferably each carrier should carry between 3-15 peptides.

In an embodiment of the invention a preferred carrier is Protein D from Haemophilus influenzae (EP 0 594 610 B1). Protein D is an IgD-binding protein from Haemophilus influenzae and has been patented by Forsgren (WO 91/18926, granted EP 0 594 610 B1). In some circumstances, for example in recombinant immunogen expression systems it may be desirable to use fragments of protein D, for example Protein D ⅓^{rd} (comprising the N-terminal 100-110 amino acids of protein D (GB 9717953.5)).

Another preferred method of presenting the IgE peptides of the present invention is in the context of a recombinant fusion molecule. For example, EP 0 421 635 B describes the use of chimeric hepadnavirus core antigen particles to present foreign peptide sequences in a virus-like particle. As such, immunogens of the present invention may comprise IgE peptides presented in chimeric particles consisting of hepatitis B core antigen. Additionally, the recombinant fusion proteins may comprise the mimotopes of the present invention and a carrier protein, such as NS1 of the influenza virus. For any recombinantly expressed protein which forms part of the present invention, the nucleic acid which encodes said immunogen also forms an aspect of the present invention, as does an expression vector comprising the nucleic acid, and a host cell containing the expression vector (autonomously or chromosomally inserted). A method of recombinantly producing the immunogen by expressing it in the above host cell and isolating the immunogen therefrom is a further aspect of the invention. The full-length native IgE molecule or the full-length native DNA sequence encoding it are not covered by the present invention.

Peptides used in the present invention can be readily synthesised by solid phase procedures well known in the art. Suitable syntheses may be performed by utilising “T-boc” or “F-moc” procedures. Cyclic peptides can be synthesised by the solid phase procedure employing the well-known “F-moc” procedure and polyamide resin in the fully automated apparatus. Alternatively, those skilled in the art will know the necessary laboratory procedures to perform the process manually. Techniques and procedures for solid phase synthesis are described in ‘Solid Phase Peptide Synthesis: A Practical Approach’ by E. Atherton and R. C. Sheppard, published by IRL at Oxford University Press (1989). Alternatively, the peptides may be produced by recombinant methods, including expressing nucleic acid molecules encoding the mimotopes in a bacterial or mammalian cell line, followed by purification of the expressed mimotope. Techniques for recombinant expression of peptides and proteins are known in the art, and are described in Maniatis, T., Fritsch, E. F. and Sambrook et al., Molecular cloning, a laboratory manual, 2nd Ed.; Cold Spring Harbor Laboratory Press, Cold Spring Harbor, New York (1989). For any recombinantly expressed peptide which forms part of the present invention, the nucleic acid which encodes said immunogen also forms an aspect of the present invention, as does an expression vector comprising the nucleic acid, and a host cell containing the expression vector (autonomously or chromosomally inserted).

The immunogens of the present invention may comprise the peptides as previously described, including mimotopes or analogues thereof, or may be immunologically cross-reactive derivatives or fragments thereof. Also forming part of the present invention are portions of nucleic acid which encode the immunogens of the present invention or peptides, mimotopes or derivatives thereof.

The present invention, therefore, provides the use of novel epitopes or mimotopes (as defined above) in the manufacture of pharmaceutical compositions for the prophylaxis or therapy of allergies. Immunogens comprising the mimotopes or peptides of the present invention, and carrier molecules are also provided for use in vaccines for the immunoprophylaxis or therapy of allergies. Accordingly, the mimotopes, peptides or immunogens of the present invention are provided for use in medicine, and in the medical treatment or prophylaxis of allergic disease.

Vaccines of the present invention, may advantageously also include an adjuvant. Suitable adjuvants for vaccines of the present invention comprise those adjuvants that are capable of enhancing the antibody responses against the IgE peptide immunogen. Adjuvants are well known in the art (Vaccine Design—The Subunit and Adjuvant Approach, 1995, Pharmaceutical Biotechnology, Volume 6, Eds. Powell, M. F., and Newman, M. J., Plenum Press, New York and London, ISBN 0-306-44867-X). Preferred adjuvants for use with immunogens of the present invention include aluminium or calcium salts (hydroxide or phosphate).

The vaccines of the present invention will be generally administered for both priming and boosting doses. It is expected that the boosting doses will be adequately spaced, or preferably given yearly or at such times where the levels of circulating antibody fall below a desired level. Boosting doses may consist of the peptide in the absence of the original carrier molecule. Such booster constructs may comprise an alternative carrier or may be in the absence of any carrier.

In a further aspect of the present invention there is provided an immunogen or vaccine as herein described for use in medicine.

The vaccine preparation of the present invention may be used to protect or treat a mammal susceptible to, or suffering from allergies, by means of administering said vaccine via systemic or mucosal route. These administrations may include injection via the intramuscular, intraperitoneal, intradermal or subcutaneous routes; or via mucosal administration to the oral/alimentary, respiratory, genitourinary tracts. A preferred route of administration is via the transdermal route, for example by skin patches. Accordingly, there is provided a method for the treatment of allergy, comprising the administration of a peptide, immunogen, or ligand of the present invention to a patient who is suffering from or is susceptible to allergy.

The amount of protein in each vaccine dose is selected as an amount which induces an immunoprotective response without significant adverse side effects in typical vaccinees. Such amount will vary depending upon which specific immunogen is employed and how it is presented. Generally, it is expected that each dose will comprise 1-1000 μg of protein, preferably 1-500 μg, more preferably 1-100 μg, of which 1 to 50 μg is the most preferable range. An optimal amount for a particular vaccine can be ascertained by standard studies involving observation of appropriate immune responses in subjects. Following an initial vaccination, subjects may receive one or several booster immunisations adequately spaced.

In a related aspect of the present invention are ligands capable of binding to the peptides of the present invention. Example of such ligands are antibodies (or Fab fragments). Also provided are the use of the ligands in medicine, and in the manufacture of medicaments for the treatment of allergies. The term “antibody” herein is used to refer to a molecule having a useful antigen binding specificity. Those skilled in the art will readily appreciate that this term may also cover polypeptides which are fragments of or derivatives of antibodies yet which can show the same or a closely similar functionality. Such antibody fragments or derivatives are intended to be encompassed by the term antibody as used herein.

Additionally, antibodies induced in one animal by vaccination with the peptides or immunogens of the present invention, may be purified and passively administered to another animal for the prophylaxis or therapy of allergy. The peptides of the present invention may also be used for the generation of monoclonal antibody hybridomas (using know techniques e.g. Köhler and Milstein, Nature, 1975, 256, p495), humanised monoclonal antibodies or CDR grafted monoclonals, by techniques known in the art. Such antibodies may be used in passive immunoprophylaxis or immunotherapy, or be used in the identification of IgE peptide mimotopes.

As the ligands of the present invention may be used for the prophylaxis or treatment of allergy, there is provided pharmaceutical compositions comprising the ligands of the present invention.

Aspects of the present invention may also be used in diagnostic assays. For example, panels of ligands which recognise the different peptides of the present invention may be used in assaying titres of anti-IgE present in serum taken from patients. Moreover, the peptides may themselves be used to type the circulating anti-IgE. It may in some circumstances be appropriate to assay circulating anti-IgE levels, for example in atopic patients, and as such the peptides and poly/mono-clonal antibodies of the present invention may be used in the diagnosis of atopy. In addition, the peptides may be used to affinity remove circulating anti-IgE from the blood of patients before re-infusion of the blood back into the patient.

Vaccine preparation is generally described in New Trends and Developments in Vaccines, edited by Voller et al., University Park Press, Baltimore, Md., U.S.A. 1978. Conjugation of proteins to macromolecules is disclosed by Likhite, U.S. Pat. No. 4,372,945 and by Armor et al., U.S. Pat. No. 4,474,757.

The information contained within the citations referred to in this application is incorporated by reference herein.

### EXAMPLES

The following examples illustrate but do not limit the present invention.

### Example 1

Anti-IgE ELISA Tests on Mouse Sera Against fgloop1 (Carl21/SEQ ID NO: 20) & fgloop2 (Carl30/SEQ ID NO: 29) Peptides

Aim

to test if the above peptides could induce antibodies able to block the binding of circulating IgE to its high-affinity receptor, FcεRI.

The Carl21 & Carl30 peptides were synthesised with a linker peptide at their C-terminal end to form the fgloop1 (sequence: CRVTHPHLPRALMCGSK) & fgloop2 (sequence: CYQMRVTHPHLPRALMRSTCGSK) peptides. They were conjugated to BSA and evaluated for mouse immunogenicity.

Immunisation

6-8 weeks old female BALB/c mice were immunised i.m. with 25 μg of peptide-BSA conjugate formulated in an oil-in-water emulsion/3D-MPL/QS21 adjuvant on day 0, 14 and 28. Mice were bled on day 28 and 42 (day 14 post II and III injection). The group of mice immunised with fgloop2 received a fourth injection at day 49 pIII.

ELISA

Mouse sera were first tested for recognition of peptide (conjugate control) and human IgE. This was done by a classical ELISA method. Briefly, 96-well plates were coated overnight at +4° C. with 50 μl of either peptide or human IgE (at 100 μg/ml in carbonate buffer or PBS).

After washing and saturation (PBS-0.1% Tween-5% milk powder), 50 μl of mouse sera was added as two-fold serial dilution starting at 1/500. A monoclonal mouse IgG1 Ab, PT011, was used as a standard thereby permitting calculation of polyclonal anti-IgE responses as μg/ml mAb equivalents. This mAb recognises coated IgE, soluble IgE (inhibits IgE-FcεRIα interaction) and receptor-bound IgE. After 1 h incubation at 37° C., plates were washed and bound mouse antibodies were detected by a biotinylated anti-mouse Ab followed by a peroxidated streptavidin complex. Bound peroxidase was left to react with TMB (BioRad), the reaction was stopped with H₂SO₄ and read at 450-630 nm.

Sera positive for coated human IgE were tested for capacity to inhibit soluble IgE from binding to the FcεRIα. For this, FcεRIα chain (IgE-binding chain of the high-affinity receptor) was coated onto 96-well plates at 0.5 μg/ml (50 μl/well) overnight at 4° C. Plates were washed and saturated as above. A two-fold serial dilution of mouse sera (start at 1/50) was mixed with a constant, 10 μg/ml, dose of chimeric mouse/human IgE (IgE anti-NP, Serotec). This mixture was incubated 1 h at 37° C. before adding to the FcεRIα-coated plates. Bound chimeric IgE was detected by a peroxidated anti-mouse λ light chain (Boehringer) followed by TMB and H₂SO₄ as above.

In this case, a diminished optical density (as compared to chimeric IgE alone) indicates a lowered binding of IgE to the receptor, i.e. a capacity of the peptide-induced mouse sera to bind to soluble IgE and inhibit its binding to FcεRIα.

Results

Both fg loop 1 and fg loop 2 were able to induce anti-IgE responses (Table 2). 10/10 mice immunised with fg loop 2 showed an anti-IgE response, whereas only 4/10 mice immunised with fg loop 1 generated an anti-IgE response.

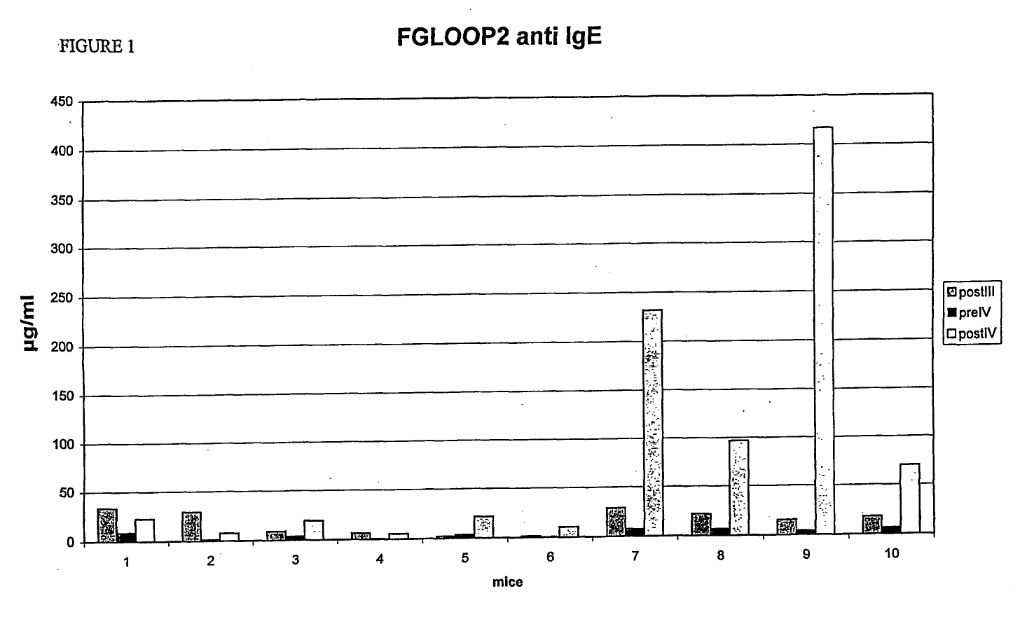

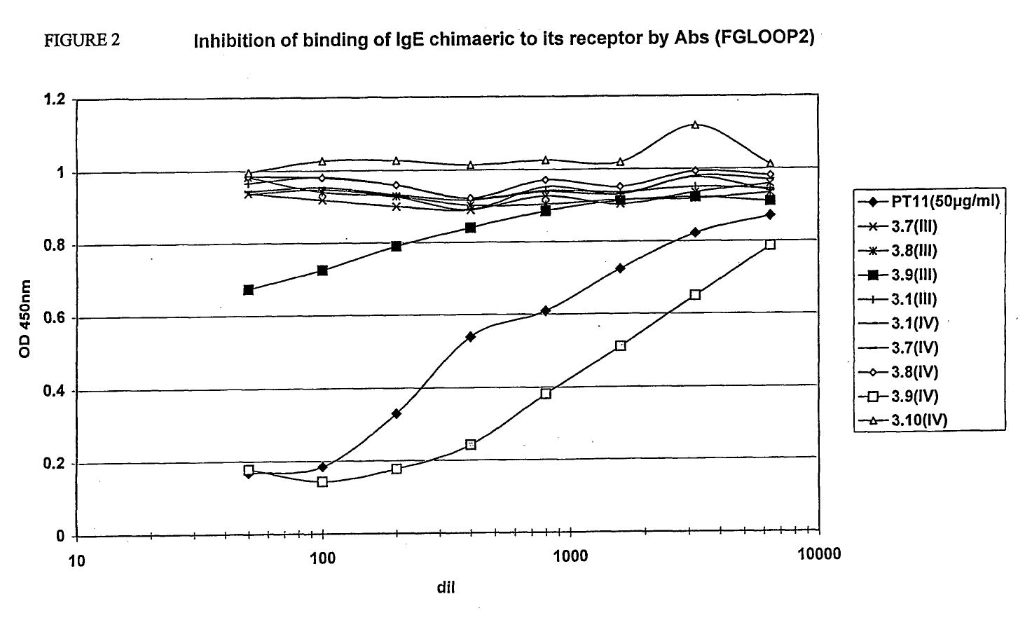

1 out of 10 mice immunised with fg loop 2 showed a high anti-IgE titre (450 μg/ml equivalent to mAb PT011) after a fourth injection (day 49 post III, bleed day 14 post IV)—see mouse 9 in FIG. 1 and Table 3. Serum from the mouse (sample 3.9) could also inhibit IgE from binding to the receptor in an ELISA setting (FIG. 2).

TABLE 2
Immune responses against fgloop1 and 2 conjugates Post III
Results Mid Point titre for anti-peptide
Results in μg/mL mAb PT11 equivalent for anti-IgE

| | Fg Loop 1 | | Fg loop2 | |
|---|---|---|---|---|
| | Peptide | IgE | Peptide | IgE |
| 1 | 16776 | — | 22342 | 42 |
| 2 | 8387 | 1.5 | 32056 | 49 |
| 3 | 18538 | — | 18034 | 15 |
| 4 | 23241 | 2.2 | 17410 | 11 |
| 5 | 27522 | 1.8 | 6441 | 1 |
| 6 | 18070 | — | 27123 | 5 |
| 7 | 18514 | 2.9 | 24683 | 49 |
| 8 | 4062 | — | 26465 | 34 |
| 9 | 4314 | — | 15609 | 19 |
| 10 | 18094 | — | 25440 | 33 |
| Average PIII | 15752 | 2.1 | 21560 | 26 |
| Average PII | 11961 | 0.6 | 14142 | 0.8 |

TABLE 3
FGLOOP2

| | | | | | anti peptide | |
|---|---|---|---|---|---|---|
| anti IgE in μg/ml equiv PT11 | | | | | (midpoint titer) | |
| | d14 postIII | d 49 postIII | d 14postIV | | 14 postIII | 14postIV |
| mice | postIII | preIV | postIV | foldincrease | postIII | postIV |
| 1 | 33 | 8 | 22 | 1 | 22342 | 82601 |
| 2 | 29 | 1 | 7 | 0 | 32056 | 27957 |
| 3 | 9 | 4 | 19 | 2 | 18034 | 23153 |
| 4 | 6 | 1 | 5 | 1 | 17410 | 14911 |
| 5 | 2 | 3 | 21 | 11 | 6441 | 29372 |
| 6 | 1 | 0 | 10 | 7 | 27123 | 37606 |
| 7 | 29 | 7 | 231 | 8 | 24683 | 28368 |
| 8 | 22 | 7 | 97 | 4 | 26465 | 37378 |
| 9 | 15 | 4 | 417 | 28 | 15609 | 48432 |
| 10 | 18 | 7 | 70 | 4 | 25440 | 51052 |
| average | 16 | 4 | 90 | 6 | 21560 | 38083 |
| stdev | 12 | 3 | 134 | 12 | 7335 | 19121 |
| geomean | 11 | 3 | 34 | 3 | 19990 | 34382 |

### Example 2

PCA Study in Rhesus Monkeys Using Mouse Sera Against fgloop2

The chimeric IgE anti-NP was mixed with two dilutions of anti-fgloop 2 mouse sera from example 1 (3.7 and 3.9=non-inhibitory and inhibitory in ELISA) or with sera against BSA conjugate without peptide (control sera). Positive control was mAb PT11.

100 μl of IgE/anti-IgE mixture was injected ID and 24 h later 6 mg of BSA-NP was injected IV to cause allergic reaction at the site of ID injection.

The wheal reaction (measured in cm) was measured about 15 min after IV injection. Two diameters were taken, one perpendicular to the other.

TABLE 4

| | | | Day 1 | | |
|---|---|---|---|---|---|
| | | | (24 hrs | | |
| | | | post IgE) | | Measured |
| | Day 0 | | IV (6 ml) | | oedema |
| Monkey 1 | ID (100 μl) | | Red-out | Hypothesised | (Length/ |
| Ri312 | (PBS-1% BSA) | | 12 minutes | oedema | wide in cm) |
| Left 1 | IgE anti-NP 100 ng | + PBS-1% BSA | BSA-NP 6 mg | +++ | 1.8/2.5 |
| 2 | IgE anti-NP 100 ng | + mAb11 10 μg | ″ | − | − |
| 3 | IgE anti-NP 100 ng | + mAb11 1 μg | ″ | −(+) | − |
| 4 | IgE anti-NP 100 ng | + IgG1 10 μg | ″ | +++ | 2.1/2.5 |
| 5 | IgE anti-NP 30 ng | + mAb11 1 μg | ″ | − | − |
| 6 | IgE anti-NP 30 ng | + IgG1 1 μg | ″ | ++ | 2.0/1.6 |
| Right 7 | IgE anti-NP 100 ng | + sera ctrl | ″ | +++ | 4.0/3.3 |
| 8 | IgE anti-NP 100 ng | + sera 3.9 high | ″ | − | − |
| 9 | IgE anti-NP 100 ng | + sera 3.9 low | ″ | −(+) | − |
| 10 | IgE anti-NP 100 ng | + sera 3.7 high | ″ | +++ | 3.2/3.0 |
| 11 | IgE anti-NP 30 ng | + sera 3.9 high | ″ | − | − |
| 12 | IgE anti-NP 30 ng | + sera ctrl | ″ | ++ | 1.8/2.0 |
| | | | Day 1 | | Measured |
| | Day 0 | | (24 hrs | | oedema |
| Monkey 2 | ID (100 μl) | | post IgE) | Hypothesised | (Length/ |
| Ri313 | (PBS-1% BSA) | | IV (6 ml) | oedema | wide in cm) |
| Left 1 | IgE anti-NP 100 ng | + PBS-1% BSA | BSA-NP 6 mg | +++ | 1.7/2.0 |
| 2 | IgE anti-NP 100 ng | + mAb11 10 μg | ″ | − | − |
| 3 | IgE anti-NP 100 ng | + mAb11 1 μg | ″ | −(+) | − |
| 4 | IgE anti-NP 100 ng | + IgG1 10 μg | ″ | +++ | 2.4/2.2 |
| 5 | IgE anti-NP 30 ng | + mAb11 1 μg | ″ | − | − |
| 6 | IgE anti-NP 30 ng | + IgG1 1 μg | ″ | ++ | 1.8/1.7 |
| Right 7 | IgE anti-NP 100 ng | + sera ctrl | ″ | +++ | 2.7/2.0 |
| 8 | IgE anti-NP 100 ng | + sera 3.9 high | ″ | − | − |
| 9 | IgE anti-NP 100 ng | + sera 3.9 low | ″ | −(+) | +/− |
| | | | | | not readible |
| 10 | IgE anti-NP 100 ng | + sera 3.7 high | ″ | +++ | 2.7/2.0 |
| 11 | IgE anti-NP 30 ng | + sera 3.9 high | ″ | − | − |
| 12 | IgE anti-NP 30 ng | + sera ctrl | ″ | ++ | 1.5/2.0 |

Rhesus monkey PCA study using the fgloop2 mouse sera of example 1 from mouse 7 (3.7) and mouse 9 (3.9). Antibody dose is indicated as final, injected dose. 100 μl is injected as a dilution in PBS-1% BSA. Both monkeys were injected identically. Experimental protocol; IgE anti-NP + anti-IgE <--------24 hrs--------> “allergen”

1. A peptide having any one of the sequences set out in SEQ ID NO: 29, 1-28, 30-34, or mimotope thereof.

2. A mimotope as claimed in claim 1, wherein the mimotope is a peptide.

3. An immunogen for the treatment of allergy comprising a peptide or mimotope as claimed in claim 1 or 2, optionally comprising a carrier molecule.

4. An immunogen as claimed in claim 3, wherein the carrier molecule is selected from Protein D or Hepatitis B core antigen.

5. An immunogen as claimed in claim 3 or 4, wherein the immunogen is a chemical conjugate of the peptide or mimotope to the carrier molecule, or wherein the immunogen is expressed as a fusion protein with the carrier molecule.

6. An immunogen as claimed in any one of claims 3 to 5, wherein the peptide or peptide mimotope is presented within the primary sequence of the carrier.

7. The immunogen of any one of claims 3-6, wherein the peptide or mimotope is structurally constrained.

8. The immunogen of claim 7, wherein the peptide or mimotope has been cyclised by a covalent bond linking its ends.

9. A vaccine comprising a peptide or a mimotope or an immunogen as claimed in any one of claims 1-8, optionally comprising an adjuvant.

10. A ligand which is capable of recognising a peptide as claimed in claim 1.

11. A pharmaceutical composition comprising a ligand as claimed in claim 10.

12. A peptide as claimed in claim 1, or immunogen as claimed in any one of claims 3-8, or vaccine as claimed in claim 9, or ligand as claimed in claim 10 for use in medicine.

13. Use of a peptide or mimotope as claimed in claim 1, or immunogen as claimed in any one of claims 3-8, or vaccine as claimed in claim 9, or ligand as claimed in claim 10 in the manufacture of a medicament for the treatment or prevention of allergy.

14. A method of manufacturing a vaccine comprising the manufacture of an immunogen as claimed in any one of claims 3 to 8, and formulating the immunogen with an excipient and/or adjuvant.

15. A method for treating a patient suffering from or susceptible to allergy, comprising the administration of a vaccine as claimed in claim 9 or a pharmaceutical composition as claimed in claim 11, to the patient.

16. An isolated nucleic acid molecule encoding the peptide of claim 1, the mimotope of claim 2, or the immunogen of any one of claims 3-6.

17. An expression vector comprising the nucleic acid molecule of claim 16.

18. A host cell comprising the expression vector of claim 17


SEQUENCE LISTING (34 sequences)

SEQ ID NO: 1 - protein, 9 aa, Homo sapiens
DSNPRGVSA

SEQ ID NO: 2 - protein, 13 aa, Artificial Sequence
  Human IgE peptide mimotope
LVVDLAPSKGTVN

SEQ ID NO: 3 - protein, 7 aa, Artificial Sequence
  Human IgE peptide mimotope
KQRNGTL

SEQ ID NO: 4 - protein, 11 aa, Artificial Sequence
  Human IgE peptide mimotope
EEKQRNGTLTV

SEQ ID NO: 5 - protein, 6 aa, Artificial Sequence
  Human IgE peptide mimotope
HPHLPR

SEQ ID NO: 6 - protein, 8 aa, Artificial Sequence
  Human IgE peptide mimotope
THPHLPRA

SEQ ID NO: 7 - protein, 10 aa, Artificial Sequence
  Human IgE peptide mimotope
VTHPHLPRAL

SEQ ID NO: 8 - protein, 12 aa, Artificial Sequence
  Human IgE peptide mimotope
RVTHPHLPRALM

SEQ ID NO: 9 - protein, 14 aa, Artificial Sequence
  Human IgE peptide mimotope
XRVTHPHLPRALMR

SEQ ID NO: 10 - protein, 16 aa, Artificial Sequence
  Human IgE peptide mimotope
QXRVTHPHLPRALMRS

SEQ ID NO: 11 - protein, 18 aa, Artificial Sequence
  Human IgE peptide mimotope
YQXRVTHPHLPRALMRST

SEQ ID NO: 12 - protein, 10 aa, Artificial Sequence
  Human IgE peptide mimotope
PEWPGSRDKR

SEQ ID NO: 13 - protein, 20 aa, Artificial Sequence
  Human IgE peptide mimotope
CDSNPRGVSAADSNPRGVSC

SEQ ID NO: 14 - protein, 15 aa, Artificial Sequence
  Human IgE peptide mimotope
CLVVDLAPSKGTVNC

SEQ ID NO: 15 - protein, 9 aa, Artificial Sequence
  Human IgE peptide mimotope
CKQRNGTLC

SEQ ID NO: 16 - protein, 13 aa, Artificial Sequence
  Human IgE peptide mimotope
CEEKQRNGTLTVC

SEQ ID NO: 17 - protein, 8 aa, Artificial Sequence
  Human IgE peptide mimotope
CHPHLPRC

SEQ ID NO: 18 - protein, 10 aa, Artificial Sequence
  Human IgE peptide mimotope
CTHPHLPRAC

SEQ ID NO: 19 - protein, 12 aa, Artificial Sequence
  Human IgE peptide mimotope
CVTHPHLPRALC

SEQ ID NO: 20 - protein, 14 aa, Artificial Sequence
  Human IgE peptide mimotope
CRVTHPHLPRALMC

SEQ ID NO: 21 - protein, 16 aa, Artificial Sequence
  Human IgE peptide mimotope
CXRVTHPHLPRALMRC

SEQ ID NO: 22 - protein, 18 aa, Artificial Sequence
  Human IgE peptide mimotope
CQXRVTHPHLPRALMRSC

SEQ ID NO: 23 - protein, 20 aa, Artificial Sequence
  Human IgE peptide mimotope
CYQXRVTHPHLPRALMRSTC

SEQ ID NO: 24 - protein, 12 aa, Artificial Sequence
  Human IgE peptide mimotope
CPEWPGSRDKRC

SEQ ID NO: 25 - protein, 9 aa, Artificial Sequence
  Human IgE peptide mimotope
CRQRNGTLC

SEQ ID NO: 26 - protein, 13 aa, Artificial Sequence
  Human IgE peptide mimotope
CEERQRNGTLTVC

SEQ ID NO: 27 - protein, 16 aa, Artificial Sequence
  Human IgE peptide mimotope
CMRVTHPHLPRALMRC

SEQ ID NO: 28 - protein, 18 aa, Artificial Sequence
  Human IgE peptide mimotope
CQMRVTHPHLPRALMRSC

SEQ ID NO: 29 - protein, 20 aa, Artificial Sequence
  Human IgE peptide mimotope
CYQMRVTHPHLPRALMRSTC

SEQ ID NO: 30 - protein, 7 aa, Artificial Sequence
  Human IgE peptide mimotope
RQRNGTL

SEQ ID NO: 31 - protein, 11 aa, Artificial Sequence
  Human IgE peptide mimotope
EERQRNGTLTV

SEQ ID NO: 32 - protein, 14 aa, Artificial Sequence
  Human IgE peptide mimotope
MRVTHPHLPRALMR

SEQ ID NO: 33 - protein, 16 aa, Artificial Sequence
  Human IgE peptide mimotope
QMRVTHPHLPRALMRS

SEQ ID NO: 34 - protein, 18 aa, Artificial Sequence
  Human IgE peptide mimotope
YQMRVTHPHLPRALMRST